(19) United States
(12) Patent Application Publication
(10) Pub. No.: US 2024/0108452 A1
(43) Pub. Date: Apr. 4, 2024

(54) IN-SITU FENESTRATION DEVICES WITH HEATED EXPANDABLE CONE

(71) Applicant: Medtronic Vascular, Inc., Santa Rosa, CA (US)

(72) Inventors: Krista Y. LEE, Galway (IE); Ciara S. GALLAGHER, Galway (IE); Garreth BOLAND-TAN, Galway (IE); Michael J. DEANE, Galway (IE)

(21) Appl. No.: 18/374,283

(22) Filed: Sep. 28, 2023

Related U.S. Application Data
(60) Provisional application No. 63/411,767, filed on Sep. 30, 2022.

(51) Int. Cl. A61F 2/07

(52) U.S. Cl. CPC A61F 2/07; A61F 2002/061

(57) ABSTRACT

An in-situ fenestration device. The device includes a sheath including a proximal end and a distal end. The device also includes an expandable cone including a proximal end, a distal end, and a body extending between the proximal end and the distal end. The expandable cone includes a heating element. The expandable cone is configured to expand from a crimped state within the sheath into an expanded state extending from the distal end of the sheath. The heating element of the expandable cone is configured to be energized with an energy source to form a fenestration in a graft material at a fenestration site of a stent graft.

### CROSS-REFERENCE TO RELATED APPLICATION

This application claims the benefit of U.S. provisional application Ser. No. 63/411,767 filed Sep. 30, 2022, the disclosure of which is hereby incorporated in its entirety by reference.

### TECHNICAL FIELD

The present disclosure relates to in-situ fenestration devices with a heated expandable cone.

### SUMMARY

In one embodiment, an in-situ fenestration device is disclosed. The device includes a sheath including a proximal end and a distal end. The device also includes an expandable cone including a proximal end, a distal end, and a body extending between the proximal end and the distal end. The expandable cone includes a heating element. The expandable cone is configured to expand from a crimped state within the sheath into an expanded state extending from the distal end of the sheath. The heating element of the expandable cone is configured to be energized with an energy source to form a fenestration in a graft material at a fenestration site of a stent graft.

In another embodiment, an in-situ fenestration device is disclosed. The device includes a sheath including a proximal end and a distal end. The device also includes an expandable cone including a proximal end, a distal end, and a body extending between the proximal end and the distal end. The expandable cone includes a heating element. The expandable cone is configured to expand from a crimped state within the sheath into an expanded state extending from the distal end of the sheath. The device further includes an energy source configured to energize the heating element of the expandable cone to form a fenestration in a graft material at a fenestration site of a stent graft and a fenestrated material. The device also includes a barb configured to gather and to remove the fenestrated material.

In yet another embodiment, a method of forming a fenestration in a graft material at a fenestration site of a stent graft is disclosed. The method includes delivering an expandable cone in a crimped state within a sheath to the fenestration site. The expandable cone includes a proximal end, a distal end, and a body extending between the proximal end and the distal end. The expandable cone includes a heating element. The sheath includes a proximal end and a distal end. The method further includes extending the expandable cone from the distal end of the sheath to transition the expandable cone from the crimped state into an expanded state. The method further includes energizing the heating element of the expandable cone in the expanded state with an energy source to form the fenestration in the graft material at the fenestration site of the stent graft and fenestrated material.

### BRIEF DESCRIPTION OF THE DRAWINGS

FIG. 1A depicts a partial cut away, schematic, side view of an abdominal aorta and right and left renal arteries extending therefrom where a stent graft excludes the right and left renal arteries from blood perfusion.

FIG. 1B depicts a partial cut away, schematic, side view of an aortic arch branching into a brachiocephalic artery, a left common carotid artery, and a left subclavian artery where a stent graft excludes the left subclavian artery from blood perfusion.

FIG. 2A depicts a perspective view of a multi-armed cautery tool configured to cut an in-situ fenestration at a fenestration site on stent material of a stent graft deployed in an abdominal aorta or an aortic arch and to capture the cut material.

FIG. 2B depicts a side, partial cross sectional view of the multi-armed cautery tool shown of FIG. 2A.

FIG. 2C depicts a schematic, side vide of the multi-armed cautery tool of FIG. 2A with arms set at different, alternative fenestration angles (θ₁, θ₂, and θ₃).

FIGS. 3A and 3B are side views depicting a multi-armed cautery tool in an open position and a closed position, respectively, and relate to a first embodiment for translating a multi-armed cautery tool between open and closed positions.

FIGS. 3C and 3D are side views depicting a multi-armed cautery tool in a closed position and an open position, respectively, and relate to a second embodiment for translating a multi-armed cautery tool between open and closed positions.

FIG. 4 depicts a cross sectional view of an abdominal aorta having branch renal arteries branching therefrom and a side view of a stent graft in a deployed, expanded position where a fenestration site is located within the stent material of the stent graft which bridges an aneurysm and the branch renal arteries.

FIGS. 5A through 5F depict schematic side, partial cross sectional views of a multi-armed cautery tool in a series of deployment positions according to one embodiment.

FIG. 6 depicts a schematic, perspective view of an in-situ fenestration device including an expandable frame according to one embodiment.

FIG. 7A depicts a schematic, top view of heating elements and non-heating crown of an expandable frame in a first position.

FIG. 7B depicts a schematic, top view of heating elements and non-heating crown of the expandable frame of FIG. 7A in a second position.

FIG. 7C depicts a schematic, plan view of a fenestration incision formed by rotating the expandable frame of FIG. 7A from the first position to the second position.

FIGS. 8A through 8D depict schematic views of operation steps using the in-situ fenestration device of FIG. 6 to cut an in-situ fenestration.

FIG. 9A depicts a schematic view of an expandable core situated within a sheath in a crimped state according to one embodiment.

FIG. 9B depicts a schematic view of the expandable cone partially extending beyond the distal end of the sheath to expand expandable cone into an expanded state.

FIG. 9C depicts a schematic view of the expandable cone retracted within the sheath while capturing fenestrated material from an in-situ fenestration made by the expanded cone.

FIGS. 10A, 10B, 10C, and 10D depict schematic perspective views of a ring of an expandable cone coil in various levels of compression (i.e., 1, 1.5, 2., 2.5, respectively).

FIG. 11A depicts a schematic, top view of a dual lumen catheter including a first lumen and a second lumen according to one embodiment.

FIG. 11B depicts a schematic, cross sectional, side view of an energized tip and a barbed tip in the dual lumen catheter of FIG. 11A.

FIG. 11C depicts a schematic, cross sectional, side view of the energized tip rotating around the barbed tip as represented by an arrow to form a fenestration at a fenestration site.

FIG. 11D depicts a schematic, cross sectional, side view of the barbed tip removing fenestrated graft material from the fenestration site.

FIGS. 12A and 12B depict schematic, perspective views of an in-situ fenestration device in a delivery position and a deployment position, respectively.

FIGS. 13A and 13B depict a schematic, side view and a schematic end view, respectively, of an in-situ fenestration device in a delivery position.

FIGS. 13C and 13D depict a schematic, side view and a schematic end view, respectively, of the in-situ fenestration device in a partially deployed position.

FIGS. 13E and 13F depict a schematic, side view and a schematic end view, respectively, of the in-situ fenestration device in a fully deployed position.

### DETAILED DESCRIPTION

Embodiments of the present disclosure are described herein. It is to be understood, however, that the disclosed embodiments are merely examples and other embodiments can take various and alternative forms. The figures are not necessarily to scale; some features could be exaggerated or minimized to show details of particular components. Therefore, specific structural and functional details disclosed herein are not to be interpreted as limiting, but merely as a representative basis for teaching one skilled in the art to variously employ the embodiments. As those of ordinary skill in the art will understand, various features illustrated and described with reference to any one of the figures can be combined with features illustrated in one or more other figures to produce embodiments that are not explicitly illustrated or described. The combinations of features illustrated provide representative embodiments for typical applications. Various combinations and modifications of the features consistent with the teachings of this disclosure, however, could be desired for particular applications or implementations.

Directional terms used herein are made with reference to the views and orientations shown in the exemplary figures. A central axis is shown in the figures and described below. Terms such as “outer” and “inner” are relative to the central axis. For example, an “outer” surface means that the surfaces faces away from the central axis, or is outboard of another “inner” surface. Terms such as “radial,” “diameter,” “circumference,” etc. also are relative to the central axis. The terms “front,” “rear,” “upper” and “lower” designate directions in the drawings to which reference is made.

Unless otherwise indicated, for the delivery system the terms “distal” and “proximal” are used in the following description with respect to a position or direction relative to a treating clinician. “Distal” and “distally” are positions distant from or in a direction away from the clinician, and “proximal” and “proximally” are positions near or in a direction toward the clinician. For the stent-graft prosthesis, “proximal” is the portion nearer the heart by way of blood flow path while “distal” is the portion of the stent-graft further from the heart by way of blood flow path.

The following detailed description is merely exemplary in nature and is not intended to limit the invention or the application and uses of the invention. Although the description is in the context of treatment of blood vessels such as the aorta, coronary, carotid, and renal arteries, the invention may also be used in any other body passageways (e.g., aortic valves, heart ventricles, and heart walls) where it is deemed useful.

In-situ fenestration (ISF) has seen limited applicability to aortic stent grafts for endovascular aneurysm repair (EVAR) and thoracic endovascular aneurysm repair (TEVAR). In-situ fenestration of aortic stent grafts can be used to maintain perfusion to blood vessels (e.g., aortic side branch arteries or peripheral arteries) located in an area excluded by a stent graft. In-situ fenestration may be used to fenestrate (e.g., create a new opening or hole) in a stent graft in-situ (e.g., in the place of the stent graft) following deployment of the stent graft within a vascular system. Application of ISF has been typically limited to removing unintentional coverage of blood vessels (e.g., arteries) upon deployment of a stent graft, but has rarely been used in elective scenarios.

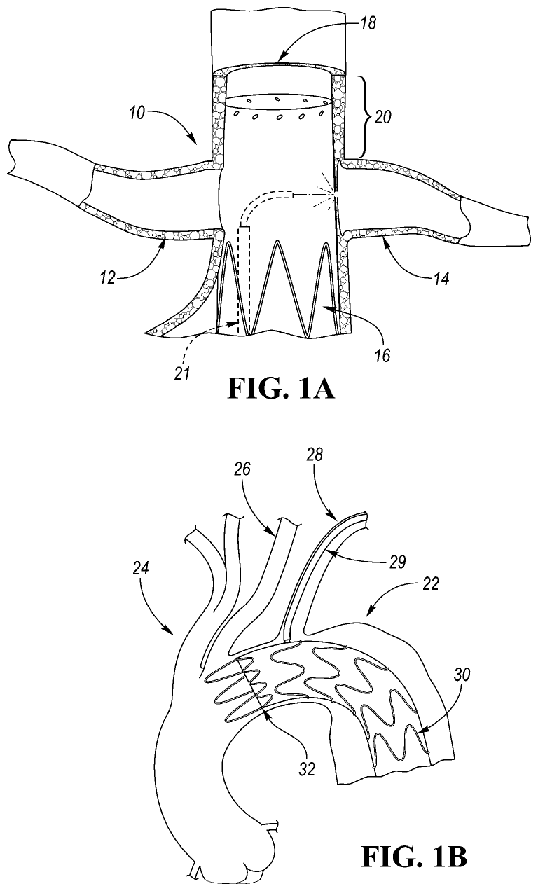

FIG. 1A depicts a partially cut away, schematic, side view of abdominal aorta 10 and right renal artery 12 and left renal artery 14 extending from abdominal aorta 10. Right and left renal arteries 12 and 14 may be referred to generally as the renal arteries. Stent graft 16 includes proximal end 18 and a distal end (not shown). Proximal end 18 of stent graft 16 lands in landing zone 20 of abdominal aorta 10. Stent graft 16 extends from landing zone 20 to exclude perfusion to right renal artery 12 and left renal artery 14. An in-situ fenestration at the exclusion areas (e.g., using laser fenestration device 21) can be used to perfuse right renal artery 12 and left renal artery 14. Perfusion may result from blood flow through the fenestration alone or through a branch stent graft inserted into the fenestration after it is created and extending into the branch artery.

FIG. 1B depicts a partial cut away, schematic, side view of aortic arch 22 branching into brachiocephalic artery 24, left common carotid artery 26, and left subclavian artery 28. Brachiocephalic artery 24, left common carotid artery 26, and left subclavian artery 28 may be referred to generally as side branch arteries. Stent graft 30 includes proximal end 32 and a distal end (not shown). Stent graft 30 extends to exclude perfusion to left subclavian artery 28. An in-situ fenestration (e.g., using laser fenestration device 29) at the exclusion area created at left subclavian artery 28 can be used to perfuse left subclavian artery 28 (e.g., via the fenestration or a later-deployed branch stent graft).

In-situ fenestration may provide a solution for implementing stent grafts with patients having hostile neck anatomy within their abdominal aorta. Current stent graft seal technology is unsuitable for many aortic anatomies. Many aortic abdominal and thoracic aortic aneurysms present either a relatively short seal zone (e.g., 0 to 10 millimeters) and/or a high degree of landing zone angulation. Examples of such anatomies include a short neck aneurysm, no neck thoraco-abdominal aneurysm, reverse conical neck, and highly angled aneurysm neck with a short landing zone inner curve. Under these circumstances, an alternative landing zone may be used that excludes perfusion to peripheral arteries (e.g., the renal arteries). In-situ fenestration may be used to open these excluded areas to permit blood perfusion. However, adequate in-situ fenestration processes and related devices/systems have not been proposed to realize the potential of in-situ fenestration in this regard.

Accordingly, clinicians (e.g., doctors or physicians) have investigated other techniques for modifying stent grafts for EVAR and TEVAR patients. The existing techniques (e.g., dedicated off-the-shelf multibranch devices, custom-made multibranch devices, clinician modified devices, and peripheral techniques) do not adequately modify stents grafts to completely address blood perfusion.

For instance, dedicated off-the-shelf multibranch devices may have low patient applicability due to variability in the anatomy of patients. The geometry to accommodate multiple branches on a dedicated branch device can be complicated to determine. Procedures to deploy these devices are complex. Branching canulation and/or stenting can be complicated because the devices are susceptible to rotational or axial misalignment.

An alternative technology is a custom-made multibranch device. However, these devices require a significant lead time (e.g., 6 to 8 weeks) and are not available for emergent cases. Moreover, custom ordered devices may still be susceptible to axial and rotational misalignment.

Clinicians have modified stent grafts themselves before deploying the stent graft in the vascular system of the patient. Physicians can partially deploy an off-the-shelf stent graft on a sterile field and make fenestrations based on patient specific anatomy. This type of “back table” modification of an off-the-shelf stent graft may have one or more benefits. Eye cautery (e.g., thermal energy) may be used to clean and/or seal any frayed and/or cut fiber ends at the fenestration boundary. The size of the fenestration is customizable without post dilation, which may cause material damage. The fenestrations can be made using three-dimensional (3D) reconstructions from patient specific computed tomography (CT) scans. The fenestrations can be reinforced with sutures and/or guidewires to make a durable interface between the main stent graft and the branch stent graft. However, these procedures include unloading of the stent graft so that it can be modified with a fenestration. Reloading the stent graft is a challenge due to the low profile and high packing density of the stent graft in the radially compressed, delivery state. These modifications are typically labor and time intensive.

Techniques for providing blood flow to peripheral blood vessels used in connection with off-the-shelf stent grafts have also been proposed. Clinicians can deploy off-the shelf stent grafts in parallel with these techniques to permit blood perfusion to peripheral arteries and respective organs. Examples of these types of technologies chimneys, snorkels, and sandwich techniques. A chimney structure may be applied in the abdominal aorta and may include a renal chimney and a seal zone distal to a lower chimney. A different structure may be applied in the aortic arch where blood flows into a chimney from the aortic arch and blood flows out of the chimney into the left common carotid artery, and blood flows into a periscope from the aortic arch and blood flows out of the periscope into the left subclavian artery. Another technique is referred to as a sandwich. Blood flows into the celiac artery and superior mesenteric artery (SMA) from sandwich parallel chimneys. These techniques may have one or more of the following benefits: (1) available for emergent cases; (2) configurations can be adapted for patient-specific anatomies (e.g., ballerina techniques); and/or (3) planning using 3D reconstructions from patient specific CT scans. However, these techniques have durability concerns and potential mid or long-term occlusion risks relating to challenging hemodynamics.

Due to one or more drawbacks of the existing technologies identified above, there has been interest in developing in-situ fenestration technology that addresses one or more of the drawbacks identified above. In-situ fenestration encompasses processes in which apertures are made in a fully or partially deployed stent graft inside of a patient. Under limited circumstances, in-situ fenestration has been employed to provide perfusion in the aortic arch, the visceral segment, and the iliac arteries. In the aortic arch, in-situ fenestration can be made in a retrograde direction (e.g., outside of the stent graft) using supra-aortic access. Other anatomies may use in situ fenestration using an antegrade technique (e.g., inside the stent graft). In-situ fenestration may have one or more of the following benefits: (1) provides a multibranch solution independent of patient anatomical constraints thus providing for a larger applicability; (2) can be performed using off-the-shelf stent grafts; and/or (3) may avoid time-consuming “back-table” modification and technically challenging reloading into delivery systems.

However, current in-situ fenestration techniques suffer from one or more drawbacks. Current in-situ fenestration methods result in relatively small size apertures where aggressive post-dilation is used to accommodate a branch stent graft. Needle in-situ fenestration uses a needle to create an initial fenestration. Laser fenestration uses a laser ablation catheter having a diameter of 2.0 to 2.5 millimeters. Radio frequency (RF) ablation may also be used. One example of an RF ablation method uses a 0.035 inch powered wire. As a drawback, damage to the graft material during fenestration expansion adds to procedural variability and makes durability testing difficult. Additionally, lack of standardized protocols results in lack of consistency in fenestrations, thereby inhibiting consistent anticipation of intermediate and long-term durability.

In one or more embodiments, in-situ fenestration process and/or related devices are disclosed that at least partially addresses one or more of the following drawbacks and/or at least partially provides one or more of the following benefits. A potential drawback of existing technology is anatomical variability limiting patient applicability of dedicated off-the shelf branch devices. A potential benefit of in-situ fenestration is customization of off the shelf stent grafts that is independent of anatomical constraints. Custom devices have been proposed but take a relatively long time (e.g., 6-8 weeks) for manufacture and deliver, and may not be available for emergent cases. A potential benefit of in-situ fenestration is application to off-the-shelf devices with no manufacturing or shipping delays.

Another potential drawback relates to “back table” modification of off-the-shelf devices by clinicians. These modified devices are difficult to reload, limiting adoption of this method. In-situ modification of a stent graft occurs in-situ, and thereby eliminating the step of reloading the device into a delivery system. Custom and “back table” modified devices are susceptible to axial or rotational misalignment which can impact vessel canulation. Fenestrations created in-situ after the deployment of a stent graft are independent of the position of the main graft.

Current in-situ fenestration procedure lack standardization in terms of initial fenestration source and post dilation procedures. A potential benefit of standardization would be the reduction or elimination of severe post dilation steps that can cause unpredictable damage to a graft material.

Current in-situ fenestration procedures may result in cut fibers and/or ripped material. These drawbacks may represent a source of procedural variability and may limit the long-term durability and seal of the fenestration and branch stent graft interface. One or more embodiments disclose a method for sealing cut fibers that help prevent continued breakdown of the fenestration and branch stent graft interface.

Current fenestration techniques start with a small initial fenestration that is aggressively post dilated to accommodate a branch graft which can result in the tearing of the graft material. Some graft materials use cutting balloons for post dilation, which may cause additional cut fibers and material damage. One or more embodiments disclose a method and/or device for forming a fenestration in-situ of a size and shape that involves little or no post dilation and/or cutting balloons.

Power sources (e.g., laser and RF ablation) for current in-situ fenestrations may create steam bubbles and generate char particles that can pose embolic risk. One or more embodiments disclose a method and/or device to allow in-situ fenestration creation while minimizing steam bubbles and char formation.

In one or more embodiments, an in-situ fenestration device with a multi-armed fenestration tool is disclosed. The multi-armed fenestration tool includes fenestration arms collectively configured to form a fenestration at a fenestration site on graft material of a deployed stent graft. The fenestration arms may be translated between a constrained position and an expanded position. The fenestration arms may be in the constrained position during delivery of the multi-armed fenestration tool to the fenestration site. The fenestration arms may be translated to the expanded state when positioned at the fenestration site.

The fenestration arms may include tip portions collectively configured to form the fenestration. The fenestration arms may be cautery arms collectively configured to cauterize (e.g., melt) portions of the graft material at the fenestration site to form the fenestration. The cautery arms may include cautery wires. The tip portions of the cautery wires may be energized with an energy source (e.g., an electrical energy source, a resistive energy source, or a radio frequency (RF) energy source) to form a fenestration. The fenestration arms may be alternatively or additionally configured to mechanically cut through the graft material at the fenestration site. The tip portions of the fenestration arms may be vibrated at a high frequency (e.g., using an ultrasonic source) to help pierce the graft material. In one or more embodiments, the tip portions of the fenestration arms may be sharpened or pointed to cut through the graft material without electrification.

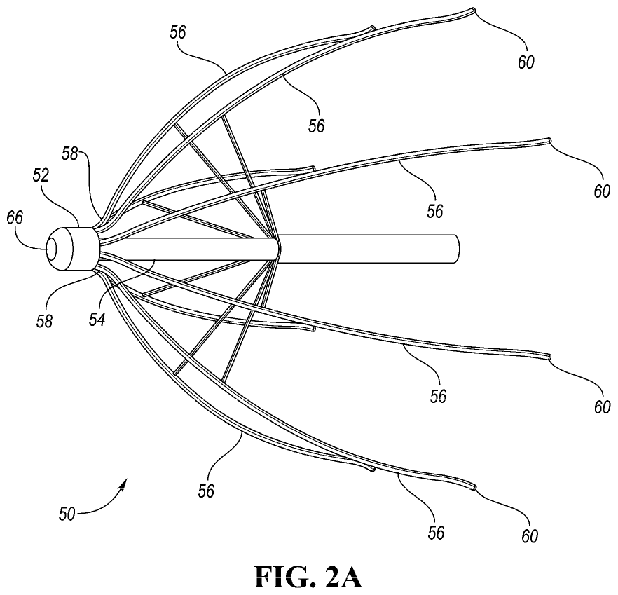

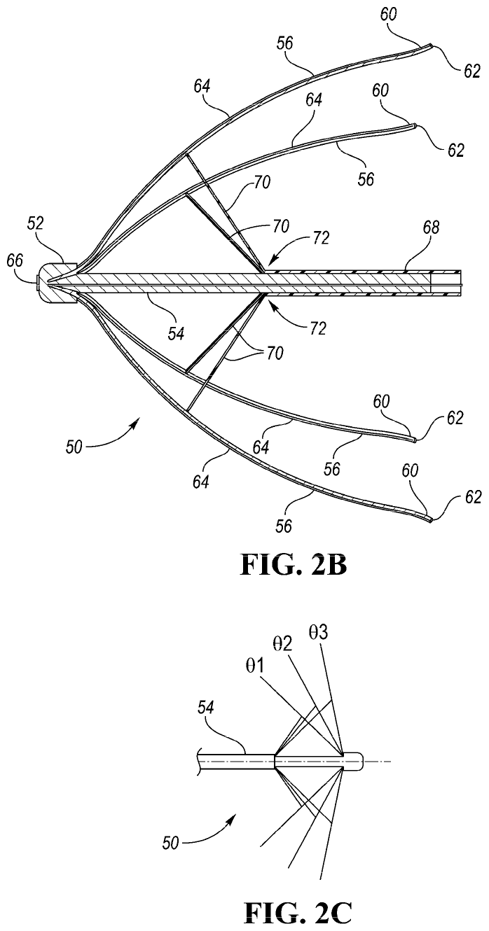

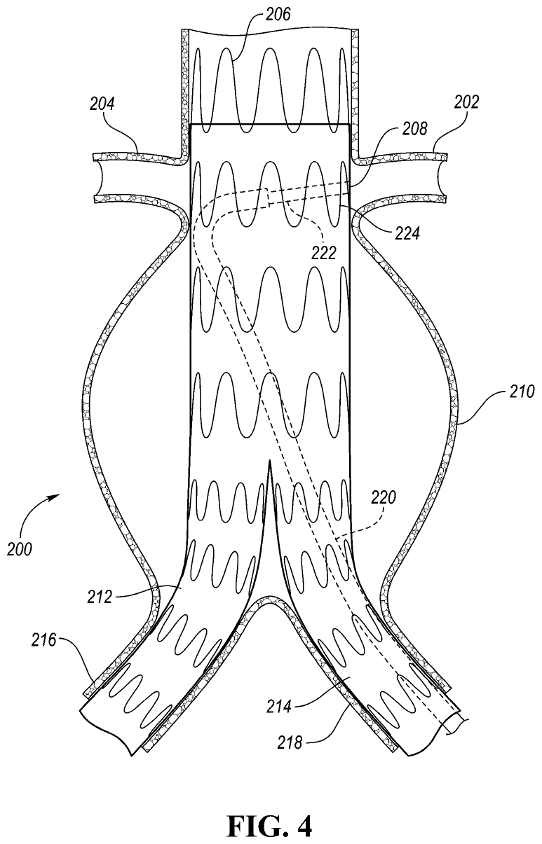

FIG. 2A depicts a perspective view of multi-armed cautery tool 50 configured to cut an in-situ fenestration at a fenestration site on stent material of a stent graft deployed in an abdominal aorta or an aortic arch and to capture the cut material. FIG. 2B depicts a side, partial cross sectional view of multi-armed cautery tool 50 shown in FIG. 2A. FIG. 2C depicts a schematic, side vide of multi-armed cautery tool 50 with arms set at different, alternative fenestration angles (θ₁, θ₂, and θ₃). Multi-armed cautery tool 50 may be delivered to the fenestration site using a steerable catheter delivery system (e.g., steerable catheter delivery system 220 as shown in FIG. 4).

The multi-armed cautery tool of one or more embodiments has one or more benefits. In one or more embodiments, the multi-armed cautery tool is configured to create an in-situ fenestration in an existing graft material. The multi-armed cautery tool may be configured to reduce or eliminate any frayed fabric after the fenestration is cut at the fenestration site. In one or more embodiments, the multi-armed cautery tool includes arms, where each arm includes a cautery wire with an insulating layer at least partially surrounding the cautery wire to protect a patient's tissue from damage from the cautery wire. The arms of the multi-armed cautery tool may be configured to capture the graft material left from the fenestration for removal from the patient's vasculature. A visualization component may be included on the multi-armed cautery tool (e.g., on the distal end thereof). The visualization component may be an echo ultrasound tip. The multi-armed cautery tool may be configured to accommodate different fenestration sizes by providing arms with adjustable arm angles relative to a central hub of the multi-armed cautery tool. In one or more embodiments, the multi-armed cautery tool may be rotated to cut the fenestration (e.g., a circular fenestration). In other embodiments, a wire may be looped through the free ends of the arms to form a wire loop configured to cut a fenestration.

As shown in FIGS. 2A and 2B, multi-armed cautery tool 50 includes distal tip 52 extending from central hub 54. Distal tip 52 may be formed of Nitinol or other shape memory material. Distal tip 52 may be a cylindrical shape, but in other embodiments, it may have a conical shape tapering inward toward the distal most end of the distal tip. Multi-armed cautery tool 50 includes arms 56 attached to and extending away from distal tip 52. The multi-armed cautery tool 50 may include any of the following number of arms or in a range of any two of these numbers: 4, 5, 6, 7, 8, 9, 10, 11, 12, 13, and 14. Arms 56 may be formed of a shape memory material (e.g., Nitinol). Arms 56 are configured to have an open position and a closed position. Arms 56 may be shape set in the open position or the closed position depending on the implementation of multi-armed cautery tool 50. As shown in FIGS. 2A and 2B, arms 56 are in in an open position where arms 56 have an outwardly tapered curved profile relative to central hub 54.

Each arm 56 includes attached end 58 and free end 60. A cautery wire may extend between attached end 58 and free end 60, with distal portion 62 of each cautery wire being exposed from insulating material 64, which may be an insulative coating (e.g., a spray or dip coating). The insulative coating may be formed of a silicone material. Insulating material 64 may be configured to protect a patient's tissue from the cautery wires.

As shown in FIGS. 2A and 2B, distal tip 52 carries cautery element 66 at a distal most end thereof. Cautery element 66 may be configured to form an initial entry hole at an in-situ fenestration site. Cautery element 66 may be energized with an energy source (e.g., an electrical energy source, a resistive energy source, or a radio frequency (RF) energy source) to form the initial entry hole. In other embodiments, distal tip 52 may include a fenestration element at the distal most end thereof. The fenestration element may be configured to mechanically cut through the graft material at the fenestration site. The fenestration element may be vibrated at a high frequency (e.g., using an ultrasonic source) to help pierce the graft material. In one or more embodiments, the fenestration element may be sharpened or pointed to cut through the graft material without electrification.

Arms 56 may be configured to be held in a closed position to form a relatively small profile of arms 56. Arms 56 may be held in the closed position during a tracking step where multi-armed cautery tool 50 tracks through steerable catheter delivery system 220 (or other suitable delivery system). Arms 56 may also be maintained in the closed position while cautery tool 50 is being positioned at the in-situ fenestration site. Once cautery tool 50 is situated at the in-situ fenestration site, arms 56 are deployed into an open position so that the fenestration can be formed in the graft material. After the fenestration is formed, arms 56 are configured to capture the cut material while in the open position. As arms 56 are translated into the closed position, the cut material from the fenestration is maintained within arms 56 with a relatively low packing density. The relatively low packing density of arms 56 in the closed position enables effective removal of the cut material from the vasculature of the patient.

As shown in FIG. 2B, central hub 54 is partially contained with central sleeve 68 to anchor central hub 54 within central sleeve 68. Central sleeve 68 may be formed of a plastic material or tube. Central hub 54 is slidable within central sleeve 68. Central sleeve 68 includes hinges or tethers 70 connected at a distal end of central sleeve 68 through hinge joints 72. Hinges 70 are also connected at central portions of arms 56. Hinges 70 may be formed of a flexible material. Hinges 70 may be configured to control the opening and closing of arms 56.

Central sleeve 68 may be connected to a locking feature formed in the handle (e.g., the proximal end of the handle). The locking feature may be configured to extend and retract central sleeve 68 relative to central hub 54 to adjust a fenestration angle, thereby adjusting the fenestration size. FIG. 2C depicts a schematic, side view of multi-armed cautery tool 50 with arms set at different, alternative fenestration angles (θ₁, θ₂, and θ₃) relative to central hub 54. A larger fenestration angle of the arms is configured to form a fenestration with a larger diameter. The θ₃ fenestration angle forms the largest diameter fenestration. The θ₁ fenestration angle forms the smallest diameter fenestration. The size of the fenestration using the θ₂ fenestration angle is between the sizes of the fenestrations using the θ₁ and θ₃ fenestration angles. The fenestration angle (e.g., the θ₁, θ₂, and θ₃ fenestration angles) and corresponding fenestration sizes mat be labelled on a delivery handle. The delivery handle may include hard stops or detents at the labelled angles and sizes for ease of use by the clinician. While three angle positions are shown, there may be fewer or greater number of angle positions, such as 1, 2, 4, or 5 positions, or any range therebetween.

In one or more embodiments, the fenestration diameter using the multi-armed cautery tool is a function of the arm length and the fenestration angle. For instance, the fenestration diameter is 8 millimeters when the arm length is 4.62 millimeters, and the fenestration angle is 60 degrees. As another example, the fenestration diameter is 4 millimeters when the arm length is 4.62 millimeters, and the fenestration angle is 25 degrees. The functional relationship between the fenestration diameter (d), the arm length (l), and the fenestration angle (θ) may be represented by the following equation (1):

d=2*l*sin θ (1)

In one or more embodiments, the fenestration diameter (d) is any of the following values or in a range of any two of the following values: 4, 5, 6, 7, or 8 millimeters. In one or more embodiments, the arm length (l) is any of the following values or in a range of any two of the following values: 2, 3, 4, 5, 6, 7, 8, 9, 10, 11, 12, 13, 14, 15, or 16 millimeters. In one or more embodiments, the fenestration angle (θ) is any of the following angles or in a range of any two of the following angles: 15, 20, 25, 30, 35, 40, 45, 50, 55, 60, 65, 70, or 75 degrees.

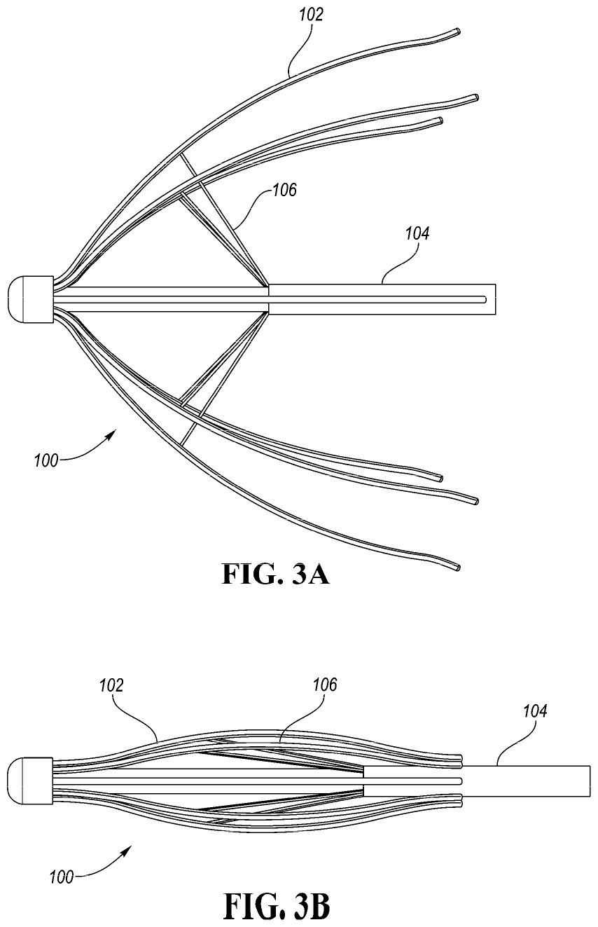

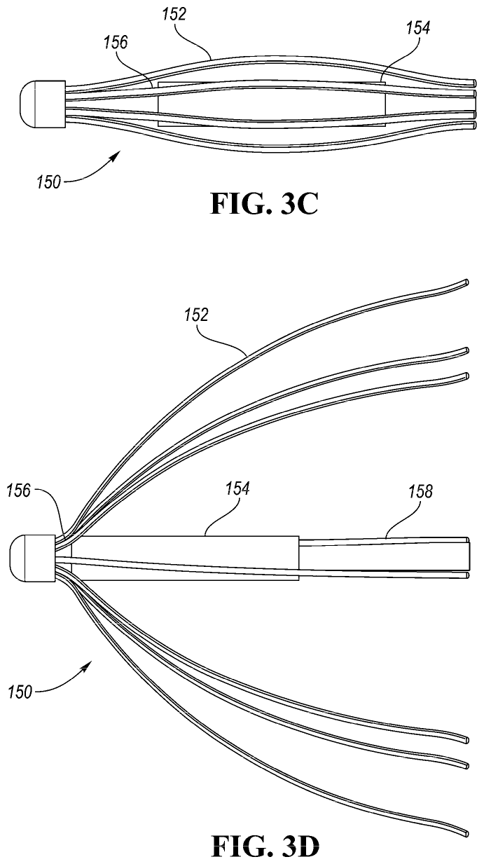

In one embodiment, the arms of the cautery tool are shape set in an open position and pulled closed by the hinges. This embodiment is depicted, for example, in FIGS. 3A and 3B. In another embodiment, the arms of the cautery tool are shape set in a football shape or oblong spheroid and are pushed open by hinges or a solid tube. This embodiment is depicted, for example, in FIGS. 3C and 3D. In the first embodiment, a material configured to fit inside a catheter and withstand a pulling force to close the arms may be used for the hinges (e.g., wires, sutures, etc.) In the second embodiment, the material used as hinges to push open the arms may be rigid but flexible (e.g., Nitinol). Alternatively, a solid metal tube formed of Nitinol or stainless steel may be used instead of hinges to push open the arms.

FIGS. 3A and 3B are side views depicting multi-armed cautery tool 100 in an open position and a closed position, respectively, and relate to a first embodiment for translating a multi-armed cautery tool between open and closed positions. Multi-armed cautery tool 100 includes arms 102. Arms 102 are memory set in the open position as shown in FIG. 3A. Arms 102 are connected to central sleeve 104 with linkages 106. As central sleeve 104 is retracted, linkages 106 pull arms 102 closed into the closed position shown in FIG. 3B. In the closed position, arms 102 have a relatively low profile and a slightly curved central portion as shown in FIG. 3B.

FIGS. 3C and 3D are side views depicting multi-armed cautery tool 150 in a closed position and an open position, respectively, and relate to a second embodiment for translating a multi-armed cautery tool between open and closed positions. Multi-armed cautery tool 150 includes arms 152. Arms 102 are memory set in the closed position as shown in FIG. 3C. Multi-armed cautery tool 150 also includes central sleeve 154. As shown in the embodiment of FIGS. 3C and 3D, central sleeve 154 and arms 152 are not connected to each other. As central sleeve 154 is advanced over central hub 158, distal end 156 of central sleeve 154 engages arms 152 and pushes them outward to translate arms 152 from the closed position to the open position. In the closed position, arms 152 have a relatively low profile and a slight curved central portion as shown in FIG. 3C.

FIG. 4 depicts a cross sectional view of abdominal aorta 200 having branch renal arteries 202 and 204 branching therefrom and a side view of stent graft 206 in a deployed, expanded position where fenestration site 208 is located within the stent material of stent graft 206 which bridges aneurysm 210 and branch renal arteries 202 and 204. Stent graft 206 includes first leg 212 and second leg 214 extending into right common iliac artery 216 and left common iliac artery 218, respectively. As shown in FIG. 4, steerable catheter delivery system 220 gains femoral access to abdominal aorta 200 through left common iliac artery 218. Steerable catheter delivery system 220 is configured to bend up to 90 degrees, for example, or any angle necessary to orient its tip perpendicular to the vessel/graft wall. As shown in FIG. 4, steerable catheter delivery system 220 is bent so that it faces fenestration site 208. Central hub 222 extends from steerable catheter delivery system 220 such that multi-armed cautery tool 224 contacts fenestration site 208.

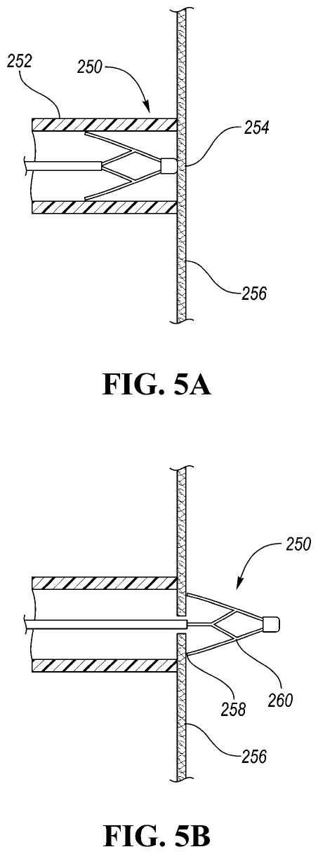

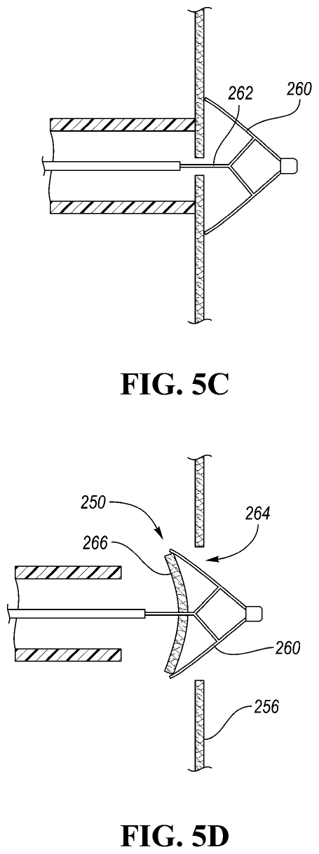

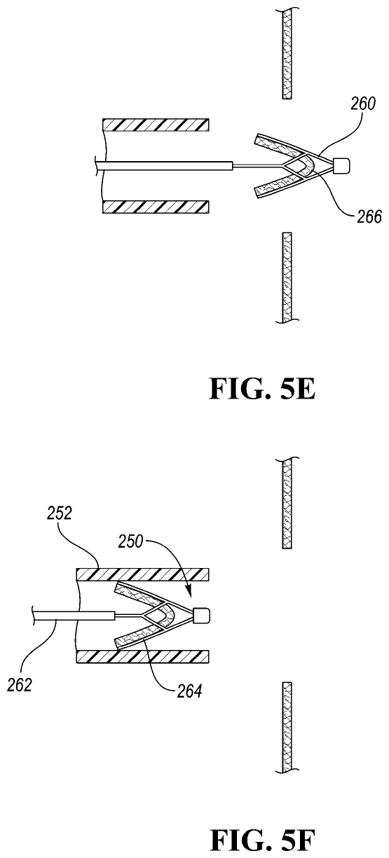

FIGS. 5A through 5F depict schematic side views of multi-armed cautery tool 250 in a series of deployment positions according to one embodiment. As shown in FIG. 5A, multi-armed cautery tool 250 is advanced through the lumen of steerable catheter delivery system 252. As shown in FIG. 5A, distal tip 254 of multi-armed cautery tool 250 includes a cautery tip at the distalmost end thereof. The cautery tip is configured to form an initial hole in graft material 256.

After the initial hole is formed in graft material 256, multi-armed cautery tool 250 is advanced through the initial hole such that multi-armed cautery tool 250 is situated within the stent graft including graft material 256. In this position, proximal ends 258 of arms 260 of multi-armed cautery tool 250 face the inner surface of graft material 256 as shown in FIG. 5B. As shown in FIG. 5B, arms 260 are in a closed position.

As shown in FIG. 5C, arms 260 are translated from the closed position to an open position. This operation may be performed as described in the embodiment shown in FIGS. 3A and 3B or the embodiment shown in FIGS. 3C and 3D. As shown in FIG. 5C, central hub 262 extends through the initial hole cut by the cautery tip.

As shown in FIG. 5D, multi-armed cautery tool 250 retracts and energized proximal ends 258 cauterize graft material 256 to form fenestration 264. The fenestrated material 266 separated from graft material 256 is disposed within the space defined within arms 260 thereby capturing the fenestrated material 266. Depending on the number, spacing, and/or size of the arms 260, the fenestrated material 266 may be separated in a single retraction step without further manipulation of the tool 250. However, if the arms are configured such that there are gaps between the openings created by the energized proximal ends where the fenestrated material 266 is still attached to the surrounding graft material, additional fenestration steps may be taken. In one example, the tool 250 may be rotated about its longitudinal axis with the energized proximal ends 258 in an activated state to cut the graft material in the gaps and complete a circular cut of the fenestrated material 266. In another example, the tool may be advanced distally, rotated, and retracted again to cut or cauterize material that was not cut in the first retraction. A physician or operator of the tool 250 may test whether a full cut has been made by applying proximal tension on the tool 250 to see if it moves freely back towards the delivery system, thereby indicating a complete cut. If there is resistance, it may mean that a further cutting step is necessary.

As shown in FIG. 5E, arms 260 are translated from the open position to the closed position, thereby maintaining fenestrated material 266 within arms 260. This operation may be performed as described in the embodiment shown in FIGS. 3A and 3B or the embodiment shown in FIGS. 3C and 3D.

As shown in FIG. 5F, central hub 262 is retracted into steerable catheter delivery system 252 to retract multi-armed cautery tool 250, which includes fenestrated material 266 therewithin. At this point, multi-armed cautery tool 250 and fenestrated material 266 may be removed from the patient's vasculature.

The multi-armed fenestration tool (e.g., cautery tool) of one or more embodiments may have one or more of the following features. The tool may change a fenestration size in-situ using an adjustable arm angle. The tool may include an insulating layer partially surrounding the wires of the cautery tool to protect a patient's tissue and vasculature. In one or more embodiments, the distal end of the cautery tool has a smooth tip to resist damage to the patient's tissue and vasculature. The distal end may have an echo tip configured for in-situ visualization of the cautery tool.

In one embodiment, an in-situ fenestration device with an expandable frame is disclosed. The expandable frame may be deployed using a relatively low-profile capsule. The expandable frame is configured to expand to a desired in-situ fenestration size. The expandable frame may include crowns including a heating element. The heating element may be a wire energized by radio frequency (RF) energy or resistive energy. In an alternative embodiment, the tips of the crowns may be sharpened or pointed to cut through graft material without heating. The tips may also be vibrated at a high frequency (e.g., ultrasonic) to help pierce the graft material.

The expandable frame in-situ fenestration device may have one or more benefits. The expandable frame may use RF technology to create a precise in-situ fenestration in a stent graft. The size of the in-situ fenestration may be adjusted by the expandable frame. The expandable frame may be formed of a shape memory material (e.g., Nitinol). The expandable frame may gradually increase in size as a longer portion of the expandable frame is deployed (e.g., advanced) from the capsule. RF energy may be activated to create an initial cut, and the expandable frame may be rotated to create the in-situ fenestration.

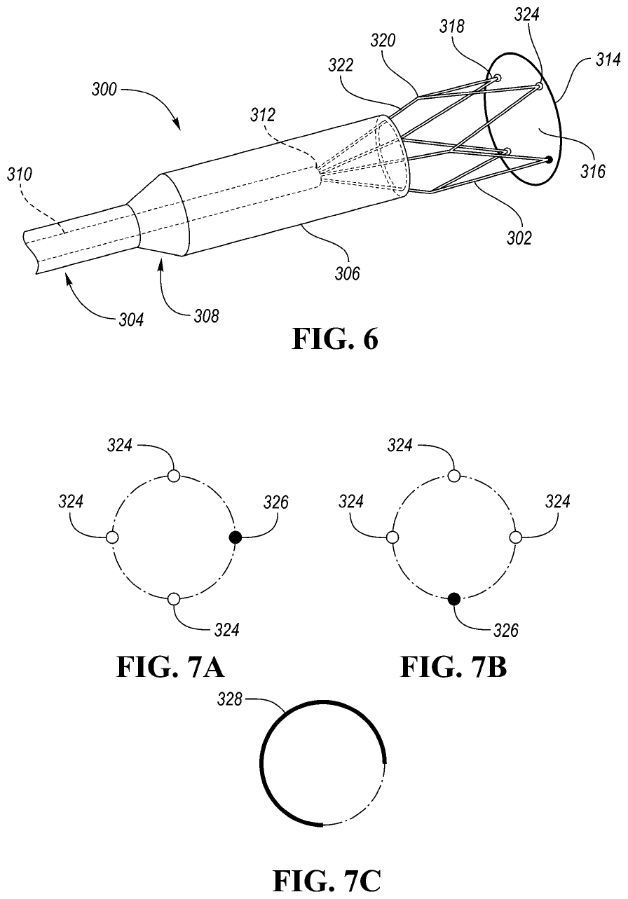

FIG. 6 depicts a schematic, perspective view of in-situ fenestration device 300 including expandable frame 302 according to one embodiment. In-situ fenestration device 300 includes outer member 304 and capsule 306 forming a distal portion of outer member 304. Capsule 306 may be bonded to the remaining portion of outer member 304. Capsule 306 has tapered portion 308 to provide a smooth transition between outer member 304 and capsule 306. In-situ fenestration device 300 also includes inner member 310. Outer member 304 and inner member 310 are configured for relative movement with respect to each other. For instance, capsule 306 may be retracted relative to inner member 310. As another example, inner member 310 may be advanced relative to capsule 306. A guide wire lumen may run through the length (e.g., the entire length) of inner member 310. An anchor ring (not shown) may be disposed proximal capsule 306. A cord or wire may be connected to anchor ring and run to a handle to permit steering of capsule 306 using the handle.

Expandable frame 302 may be bonded to distal end 312 of inner member 310 such that expandable frame 302 does not longitudinally move relative to inner member 310. Expandable frame 302 may be formed of a shape memory material, such as Nitinol. Expandable frame 302 may be shape set to an open position. As capsule 306 is retracted relative to expandable frame 302, the diameter of expandable frame 302 increases, thereby increasing the diameter of the in-situ fenestration cut with expandable frame 302. In-situ fenestration device 300 may be delivered through a branching vessel of the aortic arch. Expandable frame 302 may form in-situ fenestration 314 in graft material 316 aligned with the branching vessel and opening into the aortic arch.

Expandable frame 302 includes crowns 318, troughs 320 and struts 322 connected to troughs 320. As shown in FIG. 6, expandable frame 302 includes four crowns 318. In other embodiments, the expandable frame may have more or less crowns, for example, 3, 5, 6, 7, 8, 9, or 10 crowns. As shown in FIG. 6, expandable frame 302 is in a first partially open position. When capsule 306 is retracted further from the distal end of expandable frame 302, expandable frame 302 is in a second partially open position. Expandable frame 302 has a wider diameter in the second partially open position than in the first partially open position. When the distal ends of expandable frame 302 and capsule 306 are aligned (or the expandable frame 302 is retracted further into capsule 306), expandable frame 302 is in a closed position. The closed position may be used to deliver expandable frame 302 through a branching vessel to the desired in-situ fenestration site.

Heating elements 324 are disposed on crowns 318. Heating elements 324 may be exposed wire portions energized by RF energy. As shown in FIG. 6, three heating elements 324 are disposed on three of four crowns 318. In other embodiments, the number of heating elements may be equal to the number of crowns or less than the number of crowns (e.g., 1, 2, 3, or 4). The one or more crowns not including a heating element (e.g., non-heating crowns) do not cut through graft material but are configured to anchor expandable frame 302 against graft material 316.

FIG. 7A depicts a schematic, top view of heating elements 324 and non-heating crown 326 in a first position. FIG. 7B depicts a schematic, top view of heating elements 324 and non-heating crown 326 in a second position. FIG. 7C depicts a schematic, plan view of fenestration incision 328 formed by rotating expandable frame 302 from the first position to the second position.

Expandable frame 302 is rotated 90 degrees in a clockwise direction to move from the first position to the second position. In another embodiment, the movement may be counterclockwise to change the position of the fenestration incision. Expandable frame 302 may be rotated by rotating inner member 310, which may be rigidly connected to expandable frame 302. In one or more embodiments, inner member 310 is connected to a handle. The handle may be actuated to rotate inner member 310, which causes rotation of expandable frame 302. The rotational movement from the first position to the second position forms a 270-degree fenestration incision 328. The number of degrees of the fenestration incision may be any of the following or in a range of any two of the following: 250, 255, 260, 265, 270, 275, 280, 285, 290, 295, 300, 305, 310, 315, 320, 325, 330, 335, and 340 degrees. Non-heating crown 326 rotates through the second quadrant. Accordingly, fenestration incision 328 does not extend through the second quadrant, thereby forming an in-situ fenestration with a flap. The flap of graft material is configured to be pushed out of the way by a branching stent graft. By not creating a completely circular incision, the trapping of the cut graft material may not be necessary where the flap remains attached to the graft material. In the example shown, the flap is attached by 90 degrees of uncut graft material, however, this is merely an example. The amount of flap remaining attached may be determined by the number of crowns, their spacing, and/or the amount of rotation. As described above, there may be a range of degrees of fenestration incision and the remaining uncut portion may form the amount of attached flap material (e.g., a 300 degree cut would leave 60 degrees of attached flap).

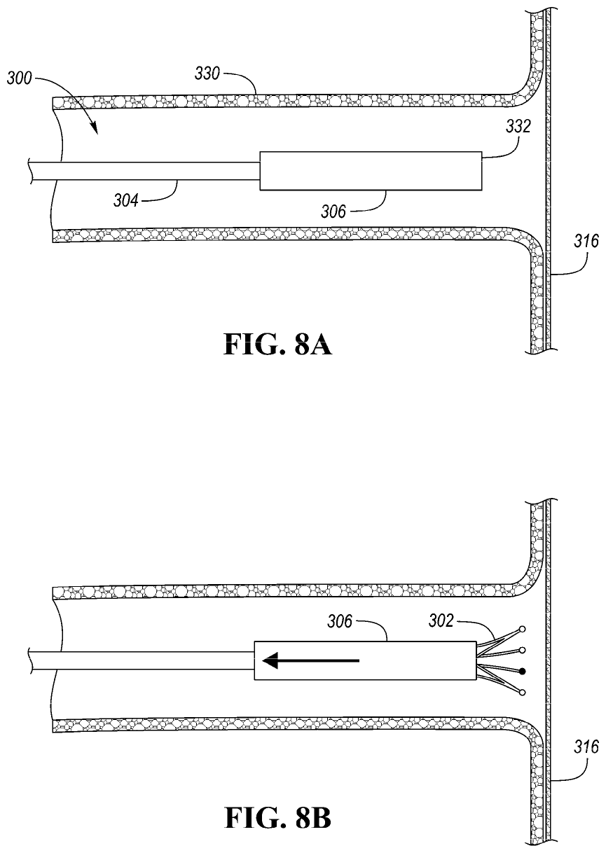

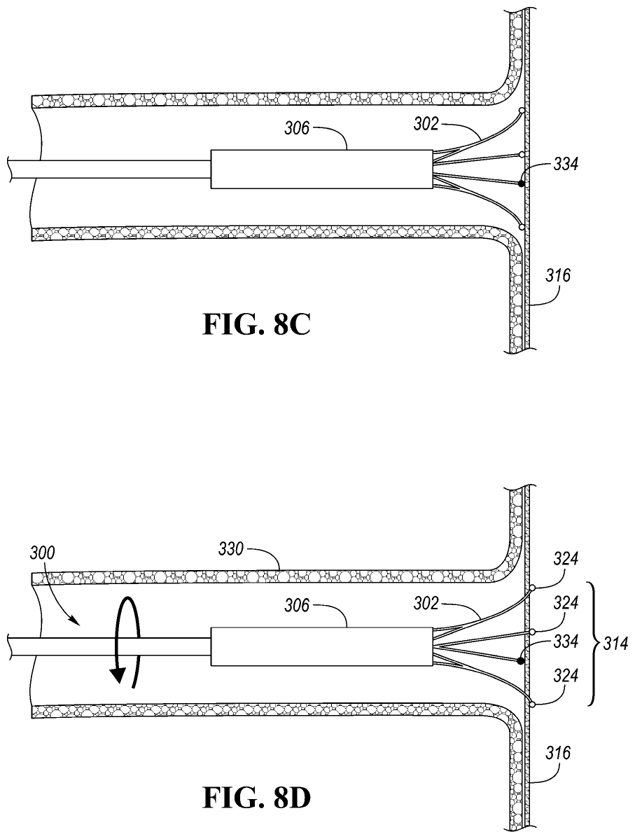

FIGS. 8A through 8D depict schematic views of operation steps using in-situ fenestration device 300 to cut in-situ fenestration 314. As shown in FIG. 8A, in-situ fenestration device 300, including outer member 304 and capsule 306, is advanced through branch vessel 330 toward graft material 316. As shown in FIG. 8A, distal end 332 of capsule 306 is spaced apart a first distance from graft material 316. As shown in FIG. 8B, capsule 306 is retracted such that it is spaced apart a second distance from graft material 316. The second distance is greater than the first distance. The retraction of capsule 306 causes expandable frame 302 to partially release and expand into a first position. As shown in FIG. 8C, distal end 334 of expandable frame 302 is advanced to contact graft material 316. The diameter of distal end 334 may be increased or decreased by retracting or advancing, respectively, capsule 306 relative to expandable frame 302. As shown in FIG. 8D, heating elements 324 are energized/activated and expandable frame 302 is rotated to cut graft material 316 to form in-situ fenestration 314. After in-situ fenestration 314 is formed, expandable frame 302 may be retracted into capsule 306 by retracting expandable frame 302 or advancing capsule 306. Once capsule 306 covers expandable frame 302, in-situ fenestration device 300 may be removed from branching vessel 330 and from the patient's vasculature.

The operation steps depicted in FIGS. 8A through 8D may be performed after the stent graft including graft material 316 is fully deployed within the aortic arch and after the delivery device for the stent graft is removed from the vasculature of a patient. Expandable frame 302 may be loaded in capsule 306 outside of the patient's vasculature (e.g., pre-loaded) and is delivered to a desired in-situ fenestration site over a guide wire. When expandable frame 302 is formed of a shape set material it expands in the heat of the patient's body. In one or more embodiments, capsule 306 may be steered to change the location of expandable frame 302. When a desired in-situ fenestration size (e.g., diameter) is deployed by adjusting the size (e.g., diameter) of the distal end of expandable frame 302, the retraction of capsule 306 may be stopped and capsule 306 may be locked relative to expandable frame 302 to prevent further relative movement. A partially deployed expandable frame 304 having the desired size may be advanced and pressed against graft material 316 prior to creating the fenestration. An RF technology may be activated to initialize the fenestration cut. Forward pressure may be applied to crowns 318 of expandable frame 302 to penetrate graft material 316 with heating elements 324. Expandable frame 302 may be rotated with heating elements 324 energized/activated to create the in-situ fenestration. In one or more embodiments, graft material 316 is not removed. Rather, in-situ fenestration device creates a flap that is configured to be pushed away by a branching graft.

In one or more embodiments, an in-situ fenestration device with a heated expandable cone is disclosed. The heated expandable cone may be formed from a metal material. The metal material at the distal end of the heated expandable cone may be heated to melt a fenestration in a graft material to a required diameter. A needle barb may be implemented to gather and to remove the fenestrated material.

Implementation of the in-situ fenestration device of one or more embodiments provides a non-invasive process of creating an in-situ fenestration. The in-situ fenestration device may be delivered via a transcatheter approach or a transfemoral approach. The fenestrated material may be captured by the in-situ fenestration device to be removed from the patient's vasculature.

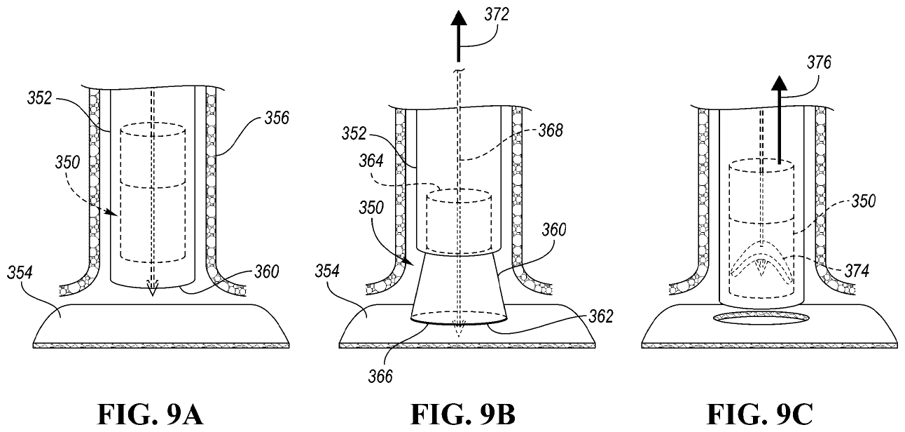

FIG. 9A depicts a schematic view of expandable cone 350 situated within sheath 352 in a crimped state. Sheath 352 maintains expandable cone 350 in the crimped state by constraining it against the inner surface of sheath 352. Expandable cone 350 may be connected to an inner catheter. The inner catheter (and therefore, expandable cone 350) may be retracted and advanced relative to sheath 352. Sheath 352, including expandable cone 350 located therein in the crimped state, may be delivered to an in-situ fenestration site on graft material 354 through branch artery 356. As shown in FIG. 9A, expanded cone 350 advances toward distal end 360 of sheath 352.

FIG. 9B depicts a schematic view of expandable cone 350 partially extending beyond distal end 360 of sheath 352 to expand expandable cone 350 into an expanded state. Expandable cone 350 may be formed of a shape memory material (e.g., Nitinol). Expandable cone 350 may be configured to be shape set into a conical shape (e.g., a truncated conical shape) where distal end 362 of expandable cone 350 has a wider diameter than proximal end 364 of expandable cone 350. The longitudinal position of expandable cone 350 may be changed to adjust the diameter of distal end 362 of expandable cone 350 as distal end 360 of sheath 352 exerts a force against the side of expandable cone 350. As shown in FIG. 9B, distal end 362 of expandable cone 350 contacts graft material 354. Distal end 362 includes heating element 366. Heating element 366 is configured to be energized with an energy source (e.g., an electrical energy source, a resistive energy source, or a radio frequency (RF) energy source) to increase the temperature of heating element 366. In one or more embodiments, heating element 366 is configured to heat up to melt the edges of an in-situ fenestration formed in graft material 354. As shown in FIG. 9B, heating element 366 is circular shaped, however other shapes may also be used. The fenestrated material formed by heating element 366 may be gathered by barb 368 (e.g., needle bard) within expandable cone 350. FIG. 9B depicts barb 368 advancing to gather fenestrated material. Arrow 372 depicts barb 368 retracting to place the fenestrated material within expandable cone 350.

FIG. 9C depicts a schematic view of expandable cone 350 retracted within sheath 352 while capturing fenestrated material 374 therein to be removed from the patient's vasculature. Arrow 376 depicts expandable cone 350 in its original, crimped state retracting relative to sheath 352.

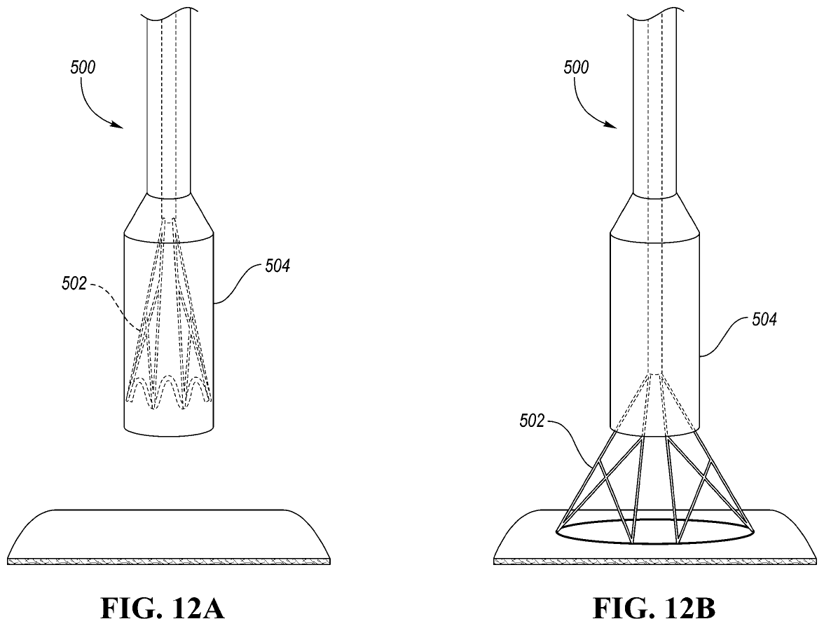

FIGS. 12A and 12B depict schematic, side views of in-situ fenestration device 500 in a delivery position and a deployment position, respectively. In-situ fenestration device 500 includes expandable cone 502, which may be formed of a solid material. The solid material may be formed of a shape setting material (e.g., a metal shape setting material). The metal shape setting material may be a combination of Nitinol and a steel alloy. The metal shape setting material may shape set into a cone shape with overlapping edges. When it is covered by sheath 504, the expandable cone 502 is encapsulated into a delivery shape as shown in FIG. 12A. When sheath 504 is retracted (or expandable cone 502 is advanced), expandable cone 502 translates into a conical, deployment shape as shown in FIG. 12B.

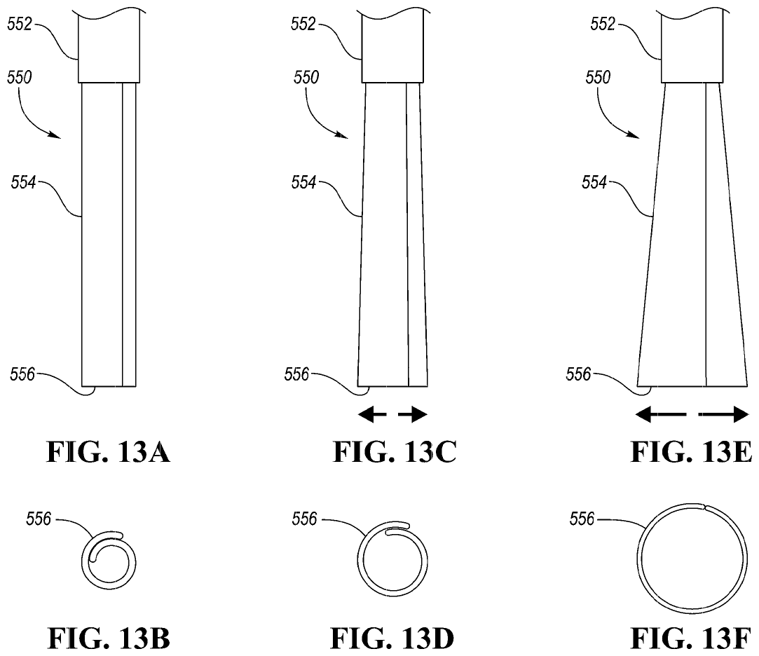

FIGS. 13A and 13B depict a schematic, side view and a schematic end view, respectively, of in-situ fenestration device 550 in a delivery position. FIGS. 13C and 13D depict a schematic, side view and a schematic end view, respectively, of in-situ fenestration device 550 in a partially deployed position. FIGS. 13E and 13F depict a schematic, side view and a schematic end view, respectively, of in-situ fenestration device 550 in a fully deployed position. In-situ fenestration device 550 includes sheath 552 and expandable cone 554. Expandable cone 554 includes distal end 556. Expandable cone 554 may be formed of a mesh material. The mesh material may be formed of a mesh shape setting material (e.g., a metal mesh shape setting material). The metal mesh shape setting material may be a Nitinol material. The metal mesh shape setting material may be shape set into a structure as shown in FIGS. 13E and 13F. When it is covered by sheath 552, expandable cone 554 is encapsulated into a cylindrical shape as shown in FIG. 13A. FIG. 13B shows an overlapping configuration of distal end 556. As sheath 552 is retracted or expandable cone 552 is advanced, distal end 556 partially unfurls as shown in FIG. 13D. In the fully deployed position in FIG. 13F, distal end 556 is in a cylindrical shape and expandable cone 554 is in a conical shape.

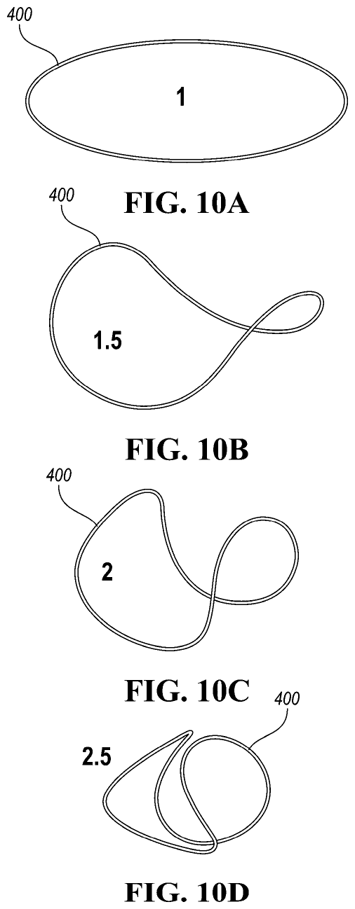

The expandable cone may include a coil with a ring configured to be compressed into a saddle shape. FIGS. 10A, 10B, 10C, and 10D depict schematic perspective views of ring 400 in various levels of compression (i.e., 1, 1.5, 2, and 2.5 respectively).

Heat may be delivered to the distal tip of the expandable cone using a soldering element (e.g., soldering iron). Heat may also be delivered using a resistive heating element or a radio frequency (RF) heating element. Heat may be delivered with wire-type electrocautery probes. The wire-type electrocautery probes are configured to reach a state of being incandescent. The wire-type electrocautery probes may reach a temperature of 500° C. or higher, which is above the melting point of materials typically used for graft material. The melting point of expanded polytetrafluorethylene (ePTFE), which is a material commonly used as a graft material, has a melting point of 327° C.

The needle barb of one or more embodiments is configured to remove fenestrated graft material and to retract via a transcatheter approach to remove the fenestrated graft material from the patient's vasculature. The needle barb may be engaged with the graft material prior to heating of the heating element to anchor the graft material, provide tension to the graft material during the fenestration process, and ensure the graft material is captured once the fenestration is made.

In one or more embodiments, a catheter including a sheath is tracked through a branch vessel pathway. Once in position at a fenestration site, an expandable cone within the sheath is deployed, thereby expanding to a conical shape. In one or more embodiments, the outer edge of the cone is heated and melts the graft material upon contact. The fenestrated material may be gathered within a needle barb and retracted out of the patient's vasculature via a central lumen. At this point in the operation, the cone may be re-sheathed and removed from the patient's body.

The in-situ fenestration device of one or more embodiments may have one or more of the following benefits. The in-situ fenestration device may be easily delivered to a desired fenestration site of a graft material to form a fenestration that is reliable and controlled. In one or more embodiments, the in-situ fenestration device implements controlled heat to result in a neat fenestration (e.g., resisting frayed edges) of the graft material.

The size of the fenestration may be adjusted in-situ by adjusting the length of the expandable cone extending from the distal end of the sheath. The fenestration size may be any of the following diameters or be in a range of any two of the following diameters: 6, 7, 8, 9, 10, 11, 12, 13, and 14 millimeters. In at least one embodiment, the fenestration may be large enough that no, or very little, post-fenestration ballooning or other dilation/expansion is necessary.

In one embodiment, an in-situ fenestration device with a rotating tip is disclosed. The in-situ fenestration device may include a dual lumen catheter containing an offset needle with a barbed tip and a radio frequency (RF) tip. The RF tip is configured to rotate about the offset needle to cut a fenestration in graft material. One or more embodiments presents a non-invasive solution that can be delivered via a transcatheter approach. The fenestrated material may be captured by the barbed tip into the dual lumen catheter and removed from the body. The tip may be activated by other forms of energy, such as resistive energy.

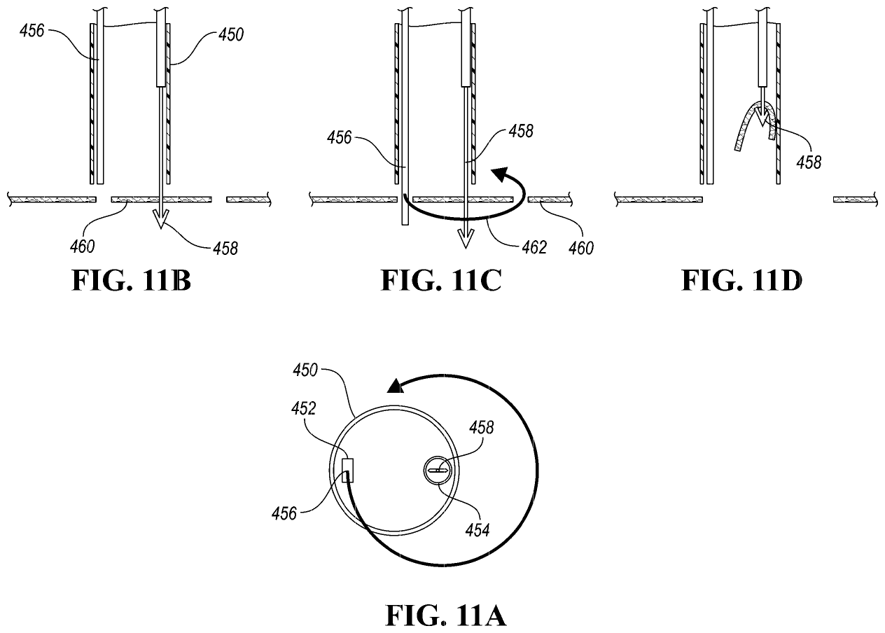

FIG. 11A depicts a schematic, top view of dual lumen catheter 450 including first lumen 452 and second lumen 454 according to one embodiment. As shown in FIG. 11A, first lumen 452 and second lumen 454 are opposite each other relative to a cross section of dual lumen catheter 450. First lumen 452 houses energized tip 456 (e.g., RF tip) and second lumen 454 houses a needle with barbed tip 458. Energized tip 456 is configured to advance through lumen 452. Needle with barbed tip 458 is configured to advance through lumen 454. Dual lumen catheter 450 may be formed of an extruded polymeric material to optimize torque performance.

FIG. 11B depicts a schematic, cross sectional, side view of energized tip 456 and barbed tip 458 in dual lumen catheter 450. As shown in FIG. 11B, barbed tip 458 is configured to pierce through and grip graft material 460 when the needle is advanced. Barbed tip 458 remains in this advanced, engaged position as energized tip 456 cuts a diameter of a fenestration. Barbed tip 458 is configured to gather the fenestrated material and retracts it through the patient's vasculature.

FIG. 11C depicts a schematic, cross sectional, side view of energized tip 456 rotating around barbed tip 458 as represented by arrow 462 to form a fenestration at a fenestration site. After barbed tip 458 pierces and grips graft material 460, energized tip 456 may be energized and advanced into graft material 460 to melt a portion of the graft material 460. Once the initial portion of the graft material 460 is melted, energized tip 456 may be rotated about the needle including barbed tip 458 that has pierced through graft material 460. The diameter of the fenestration may be any of the following diameters or in a range of any two of the following diameters: 6, 7, 8, 9, 10, 11, 12, 13, and 14 millimeters. In at least one embodiment, the fenestration may be large enough that no, or very little, post-fenestration ballooning or other dilation/expansion is necessary. The further the distance between energized tip 456 and barbed tip 458, the greater the diameter of the fenestration. Offsetting the needle from the center of the dual lumen catheter relative to an energized tip located at a peripheral portion of the dual lumen catheter results in larger fenestration while maintaining a relatively smaller catheter. The use of an energized tip forms a fenestration while reducing the likelihood of fraying.

Energized tip 456 may rotate a complete 360 degrees around barbed tip 458. In other embodiments, energized tip 456 may rotate less than 360 degrees to form a flap. The flap of graft material is configured to be pushed out of the way by a branching stent graft. By not creating a completely circular incision, the trapping of the cut graft material may not be necessary where the flap remains attached to the graft material. The number of degrees of the fenestration incision may be any of the following or in a range of any two of the following: 250, 255, 260, 265, 270, 275, 280, 285, 290, 295, 300, 305, 310, 315, 320, 325, 330, 335, 340, 345, 350, 355, and 360 degrees.

FIG. 11D depicts a schematic, cross sectional, side view of barbed tip 458 of the needle retracting through second lumen 454 to remove fenestrated graft material from the fenestration site. At this point, the fenestrated graft material may be further retracted to remove it from the patient's vasculature.

In one or more embodiments, the following procedural pathway may be used to deploy the in-situ fenestration device of one or more embodiments. Once in position at the fenestration site, a barbed tip of a needle pierces through the graft material and grips it in position. An energized tip (e.g., an RF energized tip), offset from the barbed tip, is configured to pierce/melt the graft material. Once the energized tip pierces/melts the graft material, a catheter containing a lumen housing the energized tip is rotated so that the energized tip cuts a circular incision about the needle axis (e.g., tracing a circle with the needle at the center). After the fenestration is cut, the energized tip is retracted back into the catheter. The needle and the barbed tip are also retracted, thereby collecting the graft material back into the catheter.

The in-situ fenestration device of one or more embodiments may have one or more of the following benefits. The in-situ fenestration device may be easily delivered to and removed from a desired fenestration site of a graft material to form a fenestration that is reliable and controlled.

The detailed description set forth herein includes several embodiments where each of the embodiments include several components, features, and/or steps. For the avoidance of doubt, any component, feature, and/or step of one embodiment may be applied, mixed, substituted, matched, and/or combined with one or more components, features, and/or steps of other embodiments. Such resulting embodiments are expressly within the scope of this disclosure. For example, any systems and methods for locating a branch ostium of a branch vessel disclosed herein may be used in conjunction with any disclosed embodiments. Similarly, any systems, methods, or energy types for creating a fenestration (e.g., heat, laser, vibration, RF energy, blades/mechanical cutting) may be used in any disclosed embodiments. In any of the embodiments disclosed herein, following the creation of a fenestration the fenestration may be reinforced or strengthened by placing a stent or grommet like device in the fenestration. After a fenestration is created (and optionally reinforced), a branch stent graft may be tracked and deployed within the fenestration using a separate delivery system. The branch stent graft may extend within the fenestration and at least partially within a main lumen of the fenestrated stent graft and into branch artery (e.g., renal artery, celiac, SMA, BCA, LCC, LSA, etc.). The systems, methods, and devices disclosed herein may be used to make multiple fenestrations in a single stent graft, which thereafter each receive a branch stent graft. Vacuum aspiration may be used in conjunction with any of the devices or methods described herein to remove potential emboli (e.g., gas or pieces of graft material) from the fenestration site. Vacuum aspiration may be applied through a lumen of the fenestration system or may be provided by a standalone aspiration catheter.

While exemplary embodiments are described above, it is not intended that these embodiments describe all possible forms. The words used in the specification are words of description rather than limitation, and it is understood that various changes can be made without departing from the spirit and scope of the disclosure. As previously described, the features of various embodiments can be combined to form further embodiments of the invention that may not be explicitly described or illustrated. While various embodiments could have been described as providing advantages or being preferred over other embodiments or prior art implementations with respect to one or more desired characteristics, those of ordinary skill in the art recognize that one or more features or characteristics can be compromised to achieve desired overall system attributes, which depend on the specific application and implementation. These attributes can include, but are not limited to cost, strength, durability, life cycle cost, marketability, appearance, packaging, size, serviceability, weight, manufacturability, ease of assembly, etc. As such, to the extent any embodiments are described as less desirable than other embodiments or prior art implementations with respect to one or more characteristics, these embodiments are not outside the scope of the disclosure and can be desirable for particular applications.

What is claimed is:

1. An in-situ fenestration device comprising:
a sheath including a proximal end and a distal end; and
an expandable cone including a proximal end, a distal end, and a body extending between the proximal end and the distal end, the expandable cone includes a heating element, the expandable cone is configured to expand from a crimped state within the sheath into an expanded state extending from the distal end of the sheath, and the heating element of the expandable cone is configured to be energized with an energy source to form a fenestration in a graft material at a fenestration site of a stent graft.

2. The in-situ fenestration device of claim 1, wherein the proximal end of the expandable cone has a proximal diameter and the distal end of the expandable cone has a distal diameter, the distal diameter is greater than the proximal diameter when the expandable cone is in the expanded state.

3. The in-situ fenestration device of claim 1, wherein the heating element is carried on the distal end of the expandable cone.

4. The in-situ fenestration device of claim 1, wherein the heating element has a crimped shape in the crimped state and an expanded shape in the expanded state, the crimped shape is different than the expanded shape.

5. The in-situ fenestration device of claim 4, wherein the crimped shape has a waveform profile.

6. The in-situ fenestration device of claim 4, wherein the crimped shape has an overlapping, spiral profile.

7. The in-situ fenestration device of claim 4, wherein the crimped shape has a saddle profile.

8. The in-situ fenestration device of claim 1, wherein the energy source is an electrical energy source, a resistive energy source, or a radio frequency (RF) energy source.

9. The in-situ fenestration device of claim 1, wherein the expandable cone is formed of a metal shape setting material.

10. The in-situ fenestration device of claim 4, wherein the expandable cone has a truncated conical shape in the expanded shape.

11. The in-situ fenestration device of claim 1, wherein the expandable cone is a carried on a catheter configured to retract and to advance the expandable cone relative to the sheath.

12. The in-situ fenestration device of claim 1, wherein the expandable cone is formed of a metal mesh material.

13. An in-situ fenestration device comprising:
a sheath including a proximal end and a distal end;
an expandable cone including a proximal end, a distal end, and body extending between the proximal end and the distal end, the expandable cone includes a heating element, and the expandable cone is configured to expand from a crimped state within the sheath into an expanded state extending from the distal end of the sheath;
an energy source configured to energize the heating element of the expandable cone to form a fenestration in a graft material at a fenestration site of a stent graft and a fenestrated material; and
a barb configured to gather and to remove the fenestrated material.

14. The in-situ fenestration device of claim 13, wherein the barb is a needle barb.

15. A method of forming a fenestration in a graft material at a fenestration site of a stent graft, the method comprising:
delivering an expandable cone in a crimped state within a sheath to the fenestration site, the expandable cone includes a proximal end, a distal end, and a body extending between the proximal end and the distal end, the expandable cone includes a heating element, the sheath includes a proximal end and a distal end;
extending the expandable cone from the distal end of the sheath to transition the expandable cone from the crimped state into an expanded state; and
energizing the heating element of the expandable cone in the expanded state with an energy source to form the fenestration in the graft material at the fenestration site of the stent graft and fenestrated material.

16. The method of claim 15 further comprising gathering the fenestrated material with a barb.

17. The method of claim 16 further comprising removing the fenestrated material through the sheath.

18. The method of claim 15 further comprising piercing the fenestrated material with a needle barb.

19. The method of claim 15, wherein the extending step includes retracting the sheath relative to the expandable cone.

20. The method of claim 15, wherein the extending step includes advancing the expandable cone relative to the expandable cone.
